(19) United States
(12) Patent Application Publication
(10) Pub. No.: US 2007/0248565 A1
(43) Pub. Date: Oct. 25, 2007

(54) PHARMACEUTICAL PREPARATIONS COMPRISING CORTICOSTEROIDS AND ANTIINFECTIVE AGENTS

(75) Inventors: Mohan A. CHANDAVARKAR, Bandra (IN); Nandan M. Chandavarkar, Bandra (IN); Mainde Chandrashekhar, Andheri (IN)

Correspondence Address: MERCHANT & GOULD PC, P.O. BOX 2903, MINNEAPOLIS, MN, 55402-0903, US

(73) Assignees: Chandavarkar, Mohan A., Bandra (IN); Chandavarkar, Nandan Mohan, Mumbai (IN)

(21) Appl. No.: 11/770,357

(22) Filed: Jun. 28, 2007

Related U.S. Application Data
(63) Continuation of application No. 10/416,837, filed on Oct. 20, 2003.

(30) Foreign Application Priority Data
Nov. 15, 2000 (IN) 1018/MUM/2000
Nov. 9, 2001 (IN) 1077/MUM/2001

(51) Int. Cl. A61K 31/00; A61K 31/74; A61K 9/00

(52) U.S. Cl. 424/78.04; 424/78.05; 514/169

(57) ABSTRACT

The invention consists of a stable preparation of a combination drug, comprising of an anti-inflammatory agent and an anti-infective agent. The anti-inflammatory agent in this invention is a corticosteroid, and the anti-infective agent is a derivative of quinolone, amino-glycoside or their pharmaceutically acceptable salts. The combination drug essentially comprises of i) an anti-inflammatory agent which is a corticosteroid, ii) an anti-infective agent selected from the group comprising of derivatives of quinolone, aminoglycoside and their pharmaceutically acceptable salts; iii) a complexation enhancing polymer; iv) a solubiliser exhibiting an inclusion phenomenon, along with pharmaceutically acceptable excipients with a suitable carrier system.

This application is a Continuation of application Ser. No. 10/416,837, filed May 15, 2003, and which application(s) are incorporated herein by reference.

### FIELD OF INVENTION

The invention relates to a combination drug comprising of anti-inflammatory agents in the nature of steroids and an anti-infective agent.

### BACKGROUND OF INVENTION

Anti-infective agents in the form of antibacterial agents and anti-inflammatory agents in the nature of steroids, have been used in combination for effective control of infection and inflammation. Use of such combination drugs of appropriate antibacterial agents and steroids helps in increasing patient compliance to therapy as it reduces the number of instillation of different drugs. This is specially true in the case of ophthalmic treatment, when the number of instillation of drugs requires to be minimal.

Not only is a combination of steroid and antibacterial agents desirable, but it is also essential that the combination when required for ophthalmic purposes is available as a clear formulation, either as a solution or a gel.

In formulations, for ophthalmic treatment, a clear formulation is highly desirable, because any formulation in suspension form causes irritation to the eye, as well as causes crust formation around the eye. The U.S. Pat. No. 5,472,954 describes a process by which the solubility of lipophilic or sparingly soluble active ingredients (drug! cosmetic/agrochemical) is increased by complexing with different derivatives of cyclodextrin. This patent only describes a process by which a single active ingredient can be made soluble by complexing it with cyclodextrin derivatives.

In post operative ophthalmic treatment, where in a combination therapy of antibiotic or antibacterial and anti-inflammatory agent is used, a clear formulation is highly desired. Even in non-operative cases ophthalmic preparations in clear formulation is preferred to a suspension, as a preferred line of treatment, as it increases the physical and physiological acceptance of the medication.

Ciprofloxacin, which is a quinolone derivative, and a broad spectrum antibacterial, and considered a ‘standard’ for the treatment of ocular bacterial infections is currently available in local markets in combination with the steroid dexamethasone in a suspension form. In addition U.S. Pat. No. 6,284,804 describes a suspension formulation containing dexamethasone and ciprofloxacin together with a non-ionic polymer, non-ionic surfactant and an ionic tonicity agent.

Though the above combination is known to be effective against most ocular pathogens, it is not preferred for use in post-operative cases, because it is currently available only in suspension form. An instillation of a drug in suspension form results in crust formation around the eye causing a foreign bodylike feeling in the eye and irritation. Steroids are generally sparingly soluble in aqueous solution which is the reason for most anti-infective agent/steroid combinations being available in suspension form. The antibiotic and the steroid in the form of their salts are individually available as a clear solution, but the combination, does not give a stable clear solution. On standing turbidity appears in the mixture of antibiotic steroid solution.

Further a mere admixture of antibacterial agent in clear solution and the steroid in clear solution, usually results in the crystallization of the antibacterial because of the higher pH used to dissolve the steroid, and the steroid itself degrades in a short time when mixed with the antibacterial.

The present invention describes for the first time a combination drug of a steroid and antibacterial, where in the activity and bio efficacy of the antibacterial and steroid are not in anyway hampered or affected. The invention also provides for the preparation of the combination drug in a clear solution or clear gel form, and is particularly ideal for ophthalmic treatment The clear solution or clear gels of antibacterial and steroid, would be specially suitable as a preferred form of drug administration in post operative ophthalmic cases.

### SUMMARY

In its main aspect the invention consists of a stable preparation of a combination drug, comprising of an anti inflammatory agent and an anti-infective agent. The anti-inflammatory agent in this invention is a corticosteroid, and the anti-infective agent is a derivative of quinolone, amino-glycoside or their pharmaceutically acceptable salts. The combination drug essentially comprises of i. An anti-inflammatory agent which is a corticosteroid, ii. An anti-infective agent selected from the group comprising of derivatives of quinolone, aminoglycoside and their pharmaceutically acceptable salts; iii.) a Complexation enhancing polymer; iv) a solubiliser exhibiting an inclusion phenomenon, along with pharmaceutically acceptable excipients with a suitable carrier system.

In another aspect, this invention consists of a stable pharmaceutical preparation, which is a combination drug of a corticosteroid from the group consisting of fluorometholone, dexamethasone, Beta-methasone, corticosterone, prednisolone, and their derivatives essentially having a common nuclear structure Pregna-1,4-diene-3,20 dione. The anti-infective agents are selected from the group consisting of ciprofloxacin, norfloxacin, ofloxacin, sparfloxacin, tobramycin, gentamicin and their pharmaceutically acceptable derivatives and salts.

In a further aspect of this invention, the solubiliser is selected from the group consisting of cyclodextrin and its derivatives preferably Beta cyclodextrin, and the complexation enhancing polymer used is selected from the group consisting of non-ionic polymers like polyvinyl alcohol, polyvinyl pyrrolidone, methyl cellulose or HPMC (hydroxy propyl methyl cellulose) or hydroxy ethyl cellulose.

In a more important aspect of this invention, the steroid and solubiliser are in the ratio of 1M:2M to 1M:6M.

In yet another aspect of this invention, the combination drug is incorporated in a carrier system which may be water, gel or ointment base.

In the final aspect of this invention, the combination drug when incorporated in a carrier system of water or gel, is a clear and stable pharmaceutical preparation, suitable for ocular treatment.

### DESCRIPTION

A brief description of the invention is as follows:

In its main embodiment, the invention consists of a stable pharmacuetical preparation, of a combination drug comprising of i. Anti-inflammatory agents which are corticosteroids; ii. Anti-infective agents from the group consisting of quinolone derivatives, aminoglycoside derivatives and their pharmaceutically acceptable salts; iii. A complexation enhancing polymer; and iv) a solubiliser exhibiting an inclusion phenomenon, v) pharmaceutically acceptable excipients in a carrier system

The process of manufacturing this combination drug consists of the following steps:
i. Including the steroid within a solubiliser which exhibits an inclusion phenomenon, to form a solubiliser steroid blend The steroid and solubiliser are taken in desired amounts and mixed thoroughly in a polybag to form a blend. The ratio in which the steroid and solubilizer are taken depends on the steroid and the anti-infective agent used. The solubiliser used is cyclodextrin or its derivatives, preferably Beta cyclodextrin. The use of Beta cyclodextrin makes this process cost effective.
ii. Dispersing or dissolving the anti-infective agent in a suitable solvent preferably water. The solvent and anti-infective agent are stirred to form a slurry. The complexation enhancing polymer solution is added to the slurry and sufficient water is further added and stirred for 10 to 30 minutes to form a clear anti-infective agent polymer solution. Dispersion is usually carried out at a temperature between 20° to 50° C., preferably ambient temperature. The solution is maintained at a pH range between 4 to 7.
iii. Controlled addition of anti-infective agent polymer solution to steroid solubiliser blend with constant sting to form a water soluble complex of anti-infective agent-steroid—polymer and solubiliser. The addition can even be done at room temperature. The clarity of the final solution is dependant on the crucial ratio of the anti-infective agent polymer solution to the steroid-solubiliser blend. The ratio depends upon the type of anti-infective agent and anti-inflammatory agent used. If the ratio is disturbed the clarity of the final solution is affected. The complexation usually occurs within 30 minutes. The complex formed is stable up to a temperature of 50° C.
iv. Incorporating the complex in a carrier system. The complex may be incorporated together with suitable excipients, in water, ointment base or a gel, depending upon the final purpose of its use.

The complex when dissolved in water or gel, along with excipients, forms a clear stable solution and clear stable gel respectively, which is very suitable for ocular treatment. The excipients used are benzalkonium chloride as preservative, disodium EDTA as chelating agent, mono or dibasic sodium phosphate as a buffering agent, and sodium chloride as tonicity adjustor. In respect of gel preparations sorbitol is used as tonicity adjustor.

In its preferred embodiment the steroids used are selected from the group having a general nuclear structure Pregna-1,4-diene-3,20 dione which could be dexamethasone, fluorometholone, Betamethasone, corticosterone, prednisolone and such others. The anti-infective agents are selected from the group consisting of derivatives of Ciprofloxacin, Ofloxacin, Sparfloxacin, Norfloxacin, Tobramycin sulphate, Gentamicin sulphate and their pharmceutically acceptable salts.

The following examples specifically describe the various combination drugs and the process for the different combinations of steroids and anti-infective agents used in the various carrier systems.

Manufacturing Process of the Eye Drops:

### EXAMPLE 1

Steroid—Dexamethasone

Anti-infective Agent—Ciprofloxacin Hydrochloride

1. 0.1% of Dexamethasone and 1% of Betacyclodextrin are accurately weighed.
2. Betacyclodextrin and Dexamethasone are blended thoroughly for 5 minutes.
3. 0.35% of Ciprofloxacin Hydrochloride is dispersed in water and stirred for 5 minutes to form a slurry
4. 1% of Polyvinyl alcohol solution is slowly added as a complexation enhancing agent. Sufficient water is added and stirred for 10-30 minutes to form a clear solution
5. Blend of solubilized Dexamethasone and Betacyclodextrin are gradually added to Step No. 4 by stirring it for 10-30 minutes.
6. 0.0005% of Sodium Phosphate is added to Step 5 as a buffering agent and stirred for 5 minutes.
7. 0.1% of Disodium Edetate is added to Step No. 6 as a chelating agent.
8. 0.01% of Benzalkonium Chloride is added as a preservative to Step No. 7.
9. 0.8% of Sodium Chloride is added to Step No. 8 as a tonicity adjustor.
10. The solution is stirred for 10 minutes and pH of the solution is checked. Sodium Hydroxide solution is used to adjust the pH of solution between 4.5 and 5.0. * All the steps from 1 to 9 are carried out in class 100 area.
11. The solution is filtered through 0.2μ filter to get a sterile ophthalmic solution. The solution is filled in a suitable container.

### EXAMPLE 2

Steroid—Dexamethasone

Anti-infective Agent—Tobramycin Sulphate

1. 0.1% of Dexamethasone and 1% of Betacyclodextrin are accurately weighed.
2. Betacyclodextrin and Dexamethasone are blended thoroughly for 5 minutes.
3. 0.3% of Tobramycin Sulphate is dispersed in water and stirred for 5 minutes to form a slurry
4. 1% of Polyvinyl alcohol solution is slowly added as a co-complexing agent. Sufficient water is added and stirred for 10-30 minutes to form a clear solution.
5. Blended solublized steriod of Dexamethasone and Betacyclodextrin are gradually added to Step No. 4 by stirring it for 10-30 minutes.
6. 0.0005% of Sodium Phosphate is added to Step 5 as a buffering agent.
7. 0.1% of Disodium Edetate is added to Step No. 6 as a chelating agent.
8. 0.01% of Benzalkonium Chloride is added as a preservative to Step No. 7.
9. 0.8% of Sodium Chloride is added to Step No. 8 as a tonicity adjustor.
10. The solution is stirred for 10 minutes and pH of the solution is checked. Sodium Hydroxide solution is used to adjust the pH of solution between 7.0 and 7.5. * All the steps from 1 to 9 are carried out in class 100 area.
11. The solution is filtered through 0.2μ filter to get a sterile ophthalmic solution. The solution is filled in a suitable container.

### EXAMPLE 3

Steroid—Dexamethasone

Anti-infective Agent—Ofloxacin

1. 0.1% of Dexamethasone and 1% of Betacyclodextrin are accurately weighed.
2. Betacyclodextrin and Dexamethasone are blended thoroughly for 5 minutes.
3. 0.3% of Ofloxacin is dispersed in water and stirred for 5 minutes to form a slurry
4. 1% of Polyvinyl alcohol solution is slowly added as a co-complexing agent. Sufficient water is added and stirred for 10-30 minutes to form a clear solution.
5. Blend of solublized Dexamethasone and Betacyclodextrin is gradually added to Step No. 4 by stirring it for 10-30 minutes.
6. 0.0005% of Sodium Phosphate is added to Step 5 as a buffering agent and stirred for 5 minutes.
7. 0.1% of Disodium Edetate is added to Step No. 6 as a chelating agent.
8. 0.01% of Benzalkonium Chloride is added as a preservative to Step No. 7.
9. 0.8% of Sodium Chloride is added to Step No. 8 as a tonicity adjustor.
10. The solution is stirred for 10 minutes and pH of the solution is checked. Sodium Hydroxide solution is used to adjust the pH of solution between 6.5 and 7. * All the steps from 1 to 9 are carried out in class 100 area.
11. The solution is filtered through 0.2μ filter to get a sterile ophthalmic solution. The solution is filled in a suitable container.

### EXAMPLE 4

Steroid—Dexamethasone

Anti-infective Agent—Sparfloxacin

1. 0.1% of Dexamethasone and 1% of Betacyclodextrin are accurately weighed.
2. Betacyclodextrin and Dexamethasone are blended thoroughly for 5 minutes.
3. 0.3% of Sparfloxacin is dispersed in water and stirred for 5 minutes to form a slurry
4. 1% of Polyvinyl alcohol solution is slowly added as a complexing agent. Sufficient water is added and stirred for 10-30 minutes to form a clear solution.
5. Blended solubilized steriod of Dexamethasone and Betacyclodextrin are gradually added to Step No. 4 by stirring it for 10-30 minutes.
6. 0.0005% of Sodium Phosphate is added to Step 5 as a buffering agent and stirred for 5 minutes.
7. 0.1% of Disodium Edetate is added to Step No. 6 as a chelating agent.
8. 0.01% of Benzalkonium Chloride is added as a preservative to Step No. 7.
9. 0.8% of Sodium Chloride is added to Step No. 8 as a tonicity adjustor.
10. The solution is stirred for 10 minutes and pH of the solution is checked. Sodium Hydroxide solution is used to adjust the pH; * All the steps from 1 to 9 are carried out in class 100 area.
11. The solution is filtered through 0.2μ filter to get a sterile ophthalmic solution. The solution to is filled in a suitable container.

### EXAMPLE 5

Steroid—Dexamethasone

Anti-infective Agent—Gentamicin Sulphate

1. 0.1% of Dexamethasone and 1% of Betacyclodextrin are accurately weighed.
2. Betacyclodextrin and Dexamethasone are blended thoroughly for 5 minutes.
3. 0.3% of Gentamicin Sulphate is dispersed in water and stirred for 5 minutes to form a slurry
4. 1% of Polyvinyl alcohol solution is slowly added as a co-complexing agent. Sufficient water is added and stirred for 10-30 minutes to form a clear solution.
5. Blend of solublized Dexamethasone and Betacyclodextrin is gradually added to Step No. 4 by stirring it for 10-30 minutes.
6. 0.0005% of Sodium Phosphate is added to Step 5 as a buffering agent and stirred for 5 minutes.
7. 0.1% of Disodium Edetate is added to Step No. 6 as a chelating agent
8. 0.01% of Benzalkonium Chloride is added as a preservative to Step No. 7.
9. 0.8% of Sodium Chloride is added to Step No. 8 as a tonicity adjustor.
10. The solution is stirred for 10 minutes and pH of the solution is checked. Sodium Hydroxide solution is used to adjust the pH of solution between 4.5 and 5.0. * All the steps from 1 to 9 are carried out in class 100 area.
11. The solution is filtered through 0.2μ filter to get a sterile ophthalmic solution. The solution is filled in a suitable container.

### EXAMPLE 6

Steroid: Dexamethasone

Anti-infective Agent: Norfloxacin

1. Accurately weigh 0.1% of Dexamethasone and 1% of Betacyclodextrin.
2. Blend thoroughly Betacyclodextrin and Dexamethasone for 5 minutes.
3. Disperse 0.3% of Norfloxacin in water to form a slurry and stir for 5 minutes.
4. Add 1% of Polyvinyl alcohol solution as a complexing agent slowly. Add sufficient water and stir for 10-30 minutes to form a clear solution.
5. Add solubilized steroid blend of Dexamethasone and Betacyclodextrin to Step No. 4 gradually under stirring for 10-30 minutes.
6. Add 0.0005% Sodium Phosphate as a buffering agent to Step No. 5 and stir for 5 minutes.
7. Add 0.1% of Disodium Edetate in Step No. 6 as chelating agent.
8. Add 0.01% of Benzalkonium Chloride as preservative in Step No. 7.
9. Add 0.8% of Sodium Chloride in Step No. 8 as a tonicity adjustor.
10. Stir the solution for 10 minutes and check pH of the solution. Adjust the pH of solution between 4.5 to 5.0 using Sodium Hydroxide solution. * Carry out all the steps 1 to 9 in class 100 area.
11. Filter the solution through 0.2μ filter to get a sterile ophthalmic solution. Fill the solution in suitable container.

### EXAMPLE 7

Steroid: Dexamethasone

Anti-infective Agent: Ciprofloxacin

Polymer: Polyvinyl Pyrollidone (PVP)

1. Accurately weigh 0.1% of Dexamethasone and 1% of Betacyclodextrin.
2. Blend thoroughly Betacyclodextrin and Dexamethasone for 5 minutes.
3. Disperse 0.35% of Ciprofloxacin Hydrochloride in water to form a slurry and stir for 5 minutes.
4. Add 1% of Polyvinyl Pyrollidone (PVP) solutions as a co-complexing agent slowly. Add sufficient water and stir for 10-30 minutes to form a clear solution.
5. Add solubilized blend of Dexamethasone and Betacyclodextrin to Step No. 4 gradually under stirring for 10-30 minutes.
6. Add 0.0005% Sodium Phosphate as a buffering agent to Step No. 5 and stir for 5 minutes.
7. Add 0.1% of Disodium Edetate in Step No. 6 as chelating agent.
8. Add 0.01% of Benzalkonium Chloride as preservative in Step No. 7
9. Add 0.8% of Sodium Chloride in Step No. 8 as a tonicity adjustor.
10. Stir the solution for 10 minutes and check pH of the solution. Adjust the pH of solution between 4.5 to 5.0 using Sodium Hydroxide solution. * Carry out all the steps 1 to 9 in class 100 area.
11. Filter the solution through 0.2μ filter to get a sterile ophthalmic solution. Fill the solution in suitable container.
   Manufacturing Process of Ophthalmic Gel

### EXAMPLE 8

Steroid—Dexamethasone

Anti-infective Agent—Ciprofloxacin Hydrochloride

1. 0.1% of Dexamethasone and 1% of Betacyclodextrin are accurately weighed.
2. Betacyclodextrin and Dexamethasone are blended thoroughly for 5 minutes.
3. 0.3% of Ciprofloxacin Hydrochloride is dispersed in water to form a solution and stirred for 5 minutes.
4. Blend of solublized Dexamethasone and Betacyclodextrin is gradually added by stirring it for 10-30 minutes.
5. 0.01% of Benzalkonium Chloride is dissolved as a preservative in hot water and added to Step No. 4.
6. The solution of step 5 is filtered through 0.2μ membrane filter and given a wash with water.
7. HPMC 2% is dispersed as viscofying agent in hot water with continuous stirring till a uniform gel without any lumps formation
8. 12% of sorbitol is added to Step 6 as tonicity adjustor/Humactant under constant stirring.
9. The gel is autoclaved at 121° C. for about 15 minutes.
10. The autoclaved gel is aseptically mixed (at room temperature) with the solution from Step 6 under continuous stirring till clear gel forms.
   Manufacturing Process of Ophthalmic Gel

### EXAMPLE 9

Steroid—Dexamethasone

Anti-infective Agent—Tobramycin Sulphate

1. 0.1% of Dexamethasone and 1% of Betacyclodextrin are accurately weighed.
2. Betacyclodextrin and Dexamethasone are blended thoroughly for 5 minutes.
3. 0.3% of Tobramycin Sulphate is dispersed in water to form a solution and stirred for 5 minutes.
4. Blend of solublized Dexamethasone and Betacyclodextrin is gradually added by stirring it for 10-30 minutes.
5. 0.01% of Benzalkonium Chloride is dissolved as a preservative in hot water and added to Step No. 4.
6. The solution of step 5 is filtered through 0.2μ membrane filter and given a wash with water.
7. IPMC 2% is dispersed as viscofying agent in hot water with continuous stirring till a uniform gel without any lumps formation.
8. 12% of sorbitol is added to Step 6 as tonicity adjustor/Humactant under constant stirring.
9. The gel is autoclaved at 121° C. for about 15 minutes.
10. The autoclaved gel is aseptically mixed (at room temperature) with the solution from Step 6 under continuous stirring till clear gel forms.

We claim:

1. A pharmaceutical preparation of a combination drug, comprising:
(i) an anti-infective agent, selected from the group consisting of quinolone derivatives, amino-glycoside derivatives and their pharmaceutically acceptable salts;
(ii) an anti-inflammatory agent which is a corticosteroid;
(iii) B cyclodextrin as a complexing agent and solubiliser, where in the solubiliser and steroid are in the molar ratio of 1:2 to 1:6;
(iv) a complexation enhancing polymer capable of forming a protective lubricating film;
(v) pharmaceutically acceptable excipients within a suitable carrier system; wherein the pharmaceutical preparation is stable and clear.

2. A preparation as claimed in claim 1, where in the steroid has a common nuclear structure.

3. A preparation as claimed in claim 2, where in the anti-inflammatory agent is a corticosteroid selected from the group consisting of fluorometholone, beta methasone, prednisolone, and dexamethasone.

4. A preparation as claimed in claim 3 where in the anti-infective agent is a quinolone derivative selected from the group consisting of ciprofloxacin, norfloxacin, ofloxacin, sparfloxacin, and pharmaceutically acceptable salts thereof.

5. A preparation as claimed in claim 3, where in the anti-infective agent is an aminoglycoside derivative selected from amongst tobramycin, gentamicin and its pharmaceutically acceptable salts.

6. A preparation as claimed in claim 5, where in the polymer solution is polyvinyl alcohol or polyvinyl pyrrolidone.

7. A preparation as claimed in claim 6, where in the complexing agent is β cyclodextrin.

8. A preparation as claimed in claim 7, where in the preparation is incorporated in a carrier system consisting of water and excipients resulting in a clear, stable solution for ocular treatment.

9. A preparation as claimed in claim 8 where in the preparation is incorporated in a carrier having gelling polymer and excipients resulting in a clear stable gel.

10. A preparation as claimed in claim 8 where in the preparation is incorporated in a carrier consisting of hydrophobic ointment base and excipients.

11. A preparation as claimed in claim 8, wherein the excipients used are a buffering agent, a chelating agent, a preservative, or a tonicity adjustor.

12. A preparation as claimed in claim 11, where in the excipients are benzalkonium chloride as preservatives, Edetate disodium (EDTA) as chelating agent, mono or dibasic sodium phosphate as a buffering agent and sodium chloride as tonicity adjustor, all in pharmaceutically acceptable concentrations.

13. A preparation as claimed in claim 12, where in the carrier system is water and the sodium chloride is in amounts sufficient to cause the composition to have an osmolality of about 270-320 MOSM.

14. A preparation as claimed in claim 13, wherein the concentrations of the anti-infective agent ranges from 0.3% to 0.5% wt by volume and steroid ranges from 0.1% to 0.3% wt by volume in the final preparation.

15. A method to manufacture a preparation consisting essentially of:
(a) including a steroid within a complexation forming solubilizer, to form a solubiliser-steroid blend;
(b) dispersing or dissolving an anti-infective agent in a suitable solvent, which is subsequently diluted with a solution of a complexation enhancing water soluble polymer-to give an anti-infective agent-polymer solution;
(c) combining the anti-infective agent polymer solution and steroid solubiliser blend to form a water soluble complex;
(d) Incorporating the complex in a pharmaceutically acceptable carrier system; to give a clear, stable pharmaceutical preparation.

16. A process as claimed in claim 15, wherein the solvent used for dissolving or dispersing anti-infective agent is water.

17. A process as claimed in claim 16, wherein the complexation enhancing polymer used is non-ionic, and is selected from the group consisting of polyvinyl alcohol or polyvinyl pyrrolidone.

18. A process as claimed in claim 17, where in the water soluble complex is lyophilised.

19. A process as claimed in claim 18, where in the lyophilised complex is incorporated in a suitable carrier system.

20. A process as claimed in claim 19, where in the carrier system is water with suitable excipients resulting in a clear pharmaceutical preparation for ocular treatment.

21. A process as claimed in claim 19, where in the carrier system is water, gelling polymer, and excipients.

22. A process as claimed in claim 21, where in the gelling polymer is hydroxy propy methyl cellulose, a carbome, carboxy methyl cellulose, methyl cellulose, or hydroxyethyl cellulose.

23. A process as claimed in claim 19, wherein the carrier consists of ointment base and excipients.

24. A preparation as claimed in claim 5, wherein the pharmaceutically acceptable salts are sulfates.

25. The preparation of claim 1, wherein the complexation enhancing polymer is capable of forming a protective lubricating film.
